(19) United States
(12) Patent Application Publication
(10) Pub. No.: US 2006/0019938 A1
(43) Pub. Date: Jan. 26, 2006

(54) Estrogen administration for treating male cognitive dysfunction or improving male cognitive function

(76) Inventors: Tomasz M. Beer, Portland, OR (US); Lisa B. Bland, Portland, OR (US); Mark Garzotto, Portland, OR (US); Jeri S. Janowsky, Portland, OR (US)

Correspondence Address: KLARQUIST SPARKMAN, LLP, 121 SW SALMON STREET, SUITE 1600, PORTLAND, OR, 97204, US

(21) Appl. No.: 11/027,006

(22) Filed: Dec. 30, 2004

Related U.S. Application Data
(60) Provisional application No. 60/533,693, filed on Dec. 31, 2003.
(60) Provisional application No. 60/577,054, filed on Jun. 4, 2004.

(51) Int. Cl. A61K 31/56

(52) U.S. Cl. 514/182

(57) ABSTRACT

Disclosed are methods of treating a male subject diagnosed with cognitive dysfunction that include administering an estrogenic agent, or a pharmaceutically acceptable salt or complex thereof, to the male subject. Also disclosed is a method of improving cognitive function in a hypogonadal male subject that includes transdermally administering to the male subject a therapeutically effective amount of an estrogenic agent, or a pharmaceutically acceptable salt or complex thereof. Further disclosed is a method of improving cognitive function in a male subject that includes administering to the male subject an estrogenic agent, or a pharmaceutically acceptable salt or complex thereof, at a dosage of about 0.025 to about 1.5 mg/day.

### PRIORITY CLAIM

This application claims the benefit of U.S. Provisional Application No. 60/533,693, filed Dec. 31, 2003, and U.S. Provisional Application No. 60/577,054, filed Jun. 4, 2004, both of which are incorporated herein in their entireties.

### STATEMENT OF GOVERNMENT SUPPORT

This invention was made with United States government support pursuant to Grant Nos. AG12611 and 18843 from the National Institutes of Health. The United States government has certain rights in the invention.

### FIELD

Disclosed herein are methods for treating cognitive dysfunction in male subjects and improving cognitive function in male subjects.

### BACKGROUND

As the United States, European, Canadian, Japanese and other populations age, the population of men with cognitive dysfunction, especially memory deficits, is expected to grow appreciably and therapeutic options will be needed to cope with this growing public health burden. Unfortunately, there are no established treatments that are proven to improve memory in men. Hence, methods for treating or improving cognitive function in men would be very useful.

Estrogen (usually in the form of estradiol) is often used in clinical therapy for women, for example in estrogen replacement therapy for women undergoing menopause and for treating osteoporosis. The estrogen replacement therapy dosage for treating menopause transdermally via a patch is 0.05 to 0.1 mg estradiol/day, and for oral treatment the dosage typically is 0.625 to 1.25 mg conjugated estrogen/day. The resulting mean estradiol serum concentration ranges from 60 to 100 pg/ml for the patch treatment and 25 to 50 pg/ml for oral treatment. Several studies have indicated that estrogen therapy in women may improve certain cognitive functions. See, for example, Sherwin et al., “‘Add-back’ estrogen reverses cognitive defects induced by a gonadotropin-releasing hormone agonist in women with leioyomata uteri” J. Clinical Endocrinology and metabolism 81(7), 2545-2549 (1996); and Wolf et al., “Two weeks of transdermal estradiol treatment in postmenopausal elderly women and its effect on memory and mood: Verbal memory changes are associated with the treatment induced estradiol levels,” Psychoneuroendocrinology 24:727-41, 1999. However, there is very limited information about the relationship between sex steroids and cognition in men. Men with idiopathic hypogonadism performed worse on spatial ability tests than control subjects, but men with acquired hypogonadism performed normally (Hier et al., “Spatial ability in androgen-deficient men,” N Engl J Med 306:1202-5, 1982), suggesting a developmental role for testosterone in men. Testosterone supplementation in older men improves specific aspects of cognition. See, for example, Cherrier et al., “Testosterone supplementation improves spatial and verbal memory in healthy older men,” Neurology 57:80-8, 2001; Janowsky et al., “Testosterone influences spatial cognition in older men,” Behav Neurosci 108:325-32, 1994; and Janowsky et al., “Sex steroids modify working memory,” J Cogn Neurosci 12:407-14, 2000. Thus, the conventional wisdom in the field is that only a male hormone such as testosterone may contribute to improving cognition in men.

Experimental estrogen therapy for men has only been explored for treating prostate cancer. See, for example, Ockrim et al., “Transdermal estradiol therapy for advanced prostate cancer—Forward to the past?,” J Urol 169:1735-37, 2003. Ockrim et al. is silent regarding any effects on cognition resulting from the treatment. There is no known data about the effects of estrogen supplementation on cognition in men. Moreover, the effects of such treatment on cognition cannot be reliably predicted since the influence of sex steroid hormones is gender specific in many circumstances.

### SUMMARY

Disclosed herein are methods of treating a male subject. In one variant, the method includes administering a therapeutically effective amount of an estrogenic agent, or a pharmaceutically acceptable salt or complex thereof, sufficient to achieve a supraphysiologic level of estrogenic agent in the serum of the male subject. In another variant, the method includes administering an estrogenic agent, or a pharmaceutically acceptable salt or complex thereof, at a dosage of about 0.025 mg/day to about 1.5 mg/day. In a further variant, the method includes parenterally administering a therapeutically effective amount of an estrogenic agent, or a pharmaceutically acceptable salt or complex thereof, to the male subject.

Also disclosed herein is a method of improving cognitive or emotional function in a hypogonadal male subject that includes transdermally administering to the male subject a therapeutically effective amount of an estrogenic agent, or a pharmaceutically acceptable salt or complex thereof.

In addition, disclosed herein is a method of improving cognitive or emotional function in a male subject exposed to androgen suppression therapy, by transdermally administering to the male subject a therapeutically effective amount of an estrogenic agent, or a pharmaceutically acceptable salt or complex thereof.

Another method disclosed herein involves improving cognitive function in a male subject having age-related cognitive decline by administering to the male subject a therapeutically effective amount of an estrogenic agent, or a pharmaceutically acceptable salt or complex thereof.

A further method disclosed herein involves improving cognitive or emotional function in a male subject by administering to the male subject an estrogenic agent, or a pharmaceutically acceptable salt or complex thereof, at a dosage of about 0.025 to about 1.0 mg/day.

### BRIEF DESCRIPTION OF THE FIGURES

FIGS. 1-4 are graphs showing the results of several cognitive tests performed on subjects undergoing estradiol treatment, and on control groups of test subjects.

### DETAILED DESCRIPTION OF SEVERAL EXAMPLES

For ease of understanding, the following terms used herein are described below in more detail:

“Administration of” and “administering a” compound should be understood to mean providing a compound, a prodrug of a compound, or a pharmaceutical composition as described herein. “Androgen-independent prostate cancer (AIPC)” refers to prostate cancer that progresses despite administration of primary hormonal therapy, such as androgen deprivation therapy.

An “animal” is a living multicellular vertebrate organism, a category that includes, for example, mammals and birds. A “mammal” includes both human and non-human mammals. “Subject” includes both human and animal subjects. “Cognitive dysfunction” is a condition characterized by impairment or dysfunction of at least one cognitive function such as, for example, attention, orientation, learning, memory consolidation and recall, reaction time and speech. Cognitive dysfunction can result in lowered mood and confusion among other symptoms.

“Dosage” means the amount delivered in vivo to a subject of a compound, a prodrug of a compound, or a pharmaceutical composition as described herein.

“Estrogenic agent” refers to a compound, or a pharmaceutical composition containing such compound or agent, that is, or is inclusive of, any of the sex steroid hormones (or natural or synthetic analogs thereof) referred to generically under the name “estrogen.” Such estrogenic agents generally include, but are not limited to, naturally occurring estrogens [estradiol (E₂), estrone (E), and estriol (E₃)], synthetic conjugated estrogens, polyestradiol phosphate, sulfated estrogens, and selective estrogen receptor modulators (SERMs). Illustrative specific estrogenic agents include 2-methoxyestradiol (also known as 1,3,5(10)-estratriene-3,17βdiol 2 methyl ether); 17β-estradiol (also known as estra-1,3,5(10)-triene-3,17β-diol); and SERMs such as clomiphene; cycladiene; tamoxifen; nafoxidine; nitromifene citrate (N-55,945-27); 13-ethyl-17.alpha.-ethynl-17.beta.-hydroxygona-4-9-11-trie-n-3-one (R2323); diphenol hydrochrysene; erythro-MEA; allenolic acid; cyclofenyl; chlorotrianisene; ethamoxytriphetol; triparanol; CI-626; CI-680; MER-25; U-11,555A; U-11,100A; ICI-46,669; ICI-46,474; CN-55,945; the triphenyl compounds described in U.S. Pat. No. 2,914,563; and benzothiophenes such as those described in U.S. Pat. No. 5,624,940. See, Gruber et al., “Production and actions of estrogens” N Engl J Med 346(5):340-52, 2002. “Oestrogen” is recognized as an alternative spelling for “estrogen,” and as such the term “estrogenic agent” as used herein is inclusive of oestrogen.

“Hypogonadal male” refers to a male subject that has a testosterone concentration diminished below the normal, age-appropriate range. Hypogonadal males may suffer from a disorder resulting from a variety of patho-physiological conditions in which the testosterone concentration is below normal. Hypogonadal males also may be undergoing androgen deprivation or suppression therapy such as for treating prostate cancer. The hypogonadic condition is sometimes linked with a number of physiological changes, such as diminished interest in sex, impotence, reduced lean body mass, decreased bone density, lowered mood, and energy levels.

“Mild Cognitive Impairment” (MCI) is a syndrome that was recently defined by the American Academy of Neurology. MCI refers to the clinical state of individuals who have memory impairment when compared with age appropriate normative data, but who are otherwise functioning well, and do not meet clinical criteria for dementia (see, e.g. Petersen et al., “Practice parameter: Early Detection of Dementia: Mild Cognitive Impairment,” Neurology 56:1133-42, 2001). MCI may be detected using conventional cognitive screening tests such as the Memory Impairment Screen, and various other neuropsychological screening batteries; if performance results fall outside the range of accepted normative data, MCI may be diagnosed.

“Pharmaceutically acceptable salts” of the presently disclosed estrogenic agents include those formed from cations such as sodium, potassium, aluminum, calcium, lithium, magnesium, zinc, and from bases such as ammonia, ethylenediamine, N-methyl-glutamine, lysine, arginine, omithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002).

“Pharmaceutically acceptable complexes” of the presently disclosed estrogenic agents include those complexes or coordination compounds formed from metal ions. Such complexes can include a ligand or chelating agent for bonding with an estrogenic agent.

A “pharmaceutical agent” or “drug” refers to a chemical compound or composition capable of inducing a desired therapeutic or prophylactic effect when properly administered to a subject.

“Inhibiting” means that the compounds or pharmaceutical compositions disclosed herein will slow or stop the development of disease symptoms or delay the onset of the disease.

“Supraphysiologic level” of an estrogenic agent refers to an estrogenic agent serum concentration greater than the concentration that would naturally occur in a healthy male or a hypogonadal male. For example, in a healthy male the serum concentration of estradiol ranges from 9 to 38 pg/ml. In a hypogonadal male, one study reported a median serum concentration of estradiol of 7 pg/ml (as compared to a median of 26 pg/ml in healthy men).

A “therapeutically effective amount” is an amount effective to reduce or lessen at least one symptom of the disease or condition being treated or to reduce or delay onset of one or more clinical markers or symptoms of the disease or condition.

“Transdermal” drug delivery means administration of a drug to the skin surface of an individual so that the drug passes through the skin tissue and into the individual's blood stream, thereby providing a systemic effect. The term “transdermal” is intended to include “transmucosal” drug administration, i.e., administration of a drug to the mucosal (e.g., sublingual, buccal, rectal) surface of an individual so that the drug passes through the mucosal tissue and into the individual's blood stream.

The above term descriptions are provided solely to aid the reader, and should not be construed to have a scope less than that understood by a person of ordinary skill in the art or as limiting the scope of the appended claims.

The singular terms “a,” “an,” and “the” include plural referents unless context clearly indicates otherwise. Similarly, the word “or” is intended to include “and” unless the context clearly indicates otherwise. The word “comprises” indicates “includes.” It is further to be understood that all molecular weight or molecular mass values given for compounds are approximate, and are provided for description. Although methods and materials similar or equivalent to those described herein can be used in the practice or testing of this disclosure, suitable methods and materials are described below. In addition, the materials, methods, and examples are illustrative only and not intended to be limiting. All chemical compounds disclosed herein include both the (+) and (−) stereoisomers (as well as either the (+) or (−) stereoisomer), and any tautomers thereof. An analog is a molecule that differs in chemical structure from a parent compound, for example a homolog (differing by an increment in the chemical structure, such as a difference in the length of an alkyl chain), a molecular fragment, a structure that differs by one or more functional groups, or a change in ionization. Structural analogs are often found using quantitative structure activity relationships (QSAR), with techniques such as those disclosed in Remington: The Science and Practice of Pharmacology, 19^{th} Edition (1995), chapter 28. A derivative is a biologically active molecule derived from the base structure.

Cognitive Functions that can be Treated

The presently described methods are useful for treating male subjects suffering from a condition characterized by impairment or dysfunction of at least one cognitive or emotional function and for helping to inhibit the onset of such a condition. When treating or inhibiting these conditions, the compounds or pharmaceutical compositions disclosed herein can either be used individually or in combination.

The methods described may be particularly effective for treating cognitive dysfunction associated with androgen suppression treatment, various cognitive diseases, or aging. Cognitive diseases or disorders are characterized by progressive loss of memory, cognition, reasoning, judgment and emotional stability that may gradually lead to confusion, lowered mood, profound mental deterioration and, ultimately, death. Illustrative cognitive diseases include age-related cognitive decline, Alzheimer's Disease (AD), MCI, vascular dementia, Parkinson's disease dementia, Huntington's disease, stroke, traumatic brain injury, AIDS-associated dementia, amnesic disorder and schizophrenia. The therapies described herein are particularly effective for treating memory loss and confusion. In certain examples, the methods can be used to treat patients who have been diagnosed as having or at risk of developing disorders in which diminished long-term memory is a symptom. The methods can also be used to treat normal individuals for whom improved long-term memory is desired.

The term “memory” subsumes many different processes and requires the function of many different brain areas. Memory typically is distinguished between short-term memory (also referred to as “working memory”) and long-term memory. In general, working memory is the ability to “hold in mind” and flexibly manipulate information over brief periods of time in order to make a response. Long-term memory provides declarative recall, e.g., for facts and events accessible to conscious recollection, and non-declarative recall, e.g., procedural memory of skills and operations not stored regarding time and place. A newly acquired experience initially is susceptible to various forms of disruption. With time, however, the new experience becomes resistant to disruption. This observation has been interpreted to indicate that a labile, working, short-term memory is consolidated into a more stable, long-term memory (a process sometimes referred to as “memory consolidation”). Research in recent years has provided information regarding various components of memory sufficient to identify associated brain regions. Working memory is primarily mediated by the prefrontal cortex. Declarative recall or memory is primarily mediated by the medial temporal lobe. In addition, tests have been developed, as described below, to distinguish between short-term memory and long-term memory.

Androgen suppression therapy is used for treating prostate cancer. In particular, primary hormonal therapy for prostate cancer typically involves bilateral orchiectomy or medical castration with luteinizing hormone-releasing hormone agonists or antagonists. Other methods of primary hormonal therapy may include blockage of androgen receptors with specific receptor blockers, and inhibition of steroid hormone production by agents such as ketoconazole or aminoglutethamide. The cognitive impairment that arises during or after such treatment can be ameliorated by administering an estrogenic agent either as an adjuvant to the androgen suppression therapy, or in conjunction with the androgen suppression therapy. Administration of the estrogenic agent may occur simultaneously with, or subsequent to, the androgen suppression therapy. For patients with symptomatic androgen-independent prostate cancer, administration of an estrogenic agent may improve or prevent cognitive dysfunction, and thus be a useful quality-of-life tool for managing androgen-independent prostate cancer.

As mentioned above, the treatments disclosed herein are effective in treating hypogonadal males. Researchers generally classify hypogonadism into one of three types—primary, secondary or age-related. The presently disclosed methods can effectively treat all three different types, as well as males that are hypogonadal due to prostate cancer treatment.

Primary hypogonadism includes testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high FSH and LH concentrations. However, because of testicular or other failures, the high LH concentrations are not effective at stimulating testosterone production.

Secondary hypogonadism involves an idiopathic gonadotropin or LH-releasing hormone deficiency. This type of hypogonadism includes Kaliman's Syndrome, Prader-Labhart-Willi's Syndrome, Laurence-Moon-Biedl's Syndrome, pituitary insufficiency/adenomas, Pasqualim's Syndrome, hemochromatosis, hyperprolactinemia, or pituitary-hypothalamic injury from tumors, trauma, radiation, or obesity. Because patients with secondary hypogdnadism do not demonstrate an intact feedback pathway, the lower testosterone concentrations are not associated with increased LH or FSH levels. Thus, these men have low testosterone serum levels but have gonadotropins in the normal to low range.

Third, hypogonadism may be age-related. Men experience a slow but continuous decline in average serum testosterone after approximately age 20 to 30 years. Studies in men have found that the mean testosterone value at age 70 years is approximately 60% of that at age 30 years. Because the serum concentration of SHBG increases as men age, the fall in bioavailable and free testosterone is even greater than the fall in total testosterone. Researchers have estimated that approximately 50% of healthy men between the ages of 50 and 70 have levels of bioavailable testosterone that are below the lower normal limit for younger men. Moreover, as men age, the circadian rhythm of testosterone concentration is often muted, dampened, or completely lost. The major problem with aging appears to be within the hypothalamic-pituitary unit. For example, researchers have found that with aging, LH levels do not increase despite the low testosterone levels. Regardless of the cause, these untreated testosterone deficiencies in older men may lead to a variety of physiological changes, including sexual dysfunction, decreased libido, loss of muscle mass, decreased bone density, depressed mood, and decreased cognitive function. The net result is geriatric hypogonadism, or what is commonly referred to as “male menopause.”

Diagnosing Cognitive Dysfunction/Testing Cognitive Function

Individuals can be diagnosed with cognitive dysfunction through a variety of tests. A thorough discussion of these tests can be found in Lezak, Neuropsychological Assessment, Oxford Press, New York (1995). For example, such patients may be identified through a clinical diagnosis based on symptoms of dementia or learning and memory loss.

In humans, a number of psychometric measures are useful to identify individuals with memory loss or MCI. Illustrative tests include learning and recalling of word lists, paragraphs and/or non-verbal materials. Other useful measures to determine whether an individual has memory loss or MCI include, but are not limited to, the following: clinical dementia rating scale, clinical dementia rating sum of boxes, global deterioration scale, geriatric depression scale, mini-mental state examination, dementia rating scale, Weschler Adult Intelligence scale, the Weschler Memory Scale, performance IQ, Boston naming test, controlled oral word association test, logical memory I test, logical memory II test, visual reproductions test one, visual reproductions test two, auditory verbal learning test-sum of learning trials 1 to 5, auditory verbal learning test I delayed recall/trial, the free and cued selective reminding test—sum of the performance across trials 1 to 6, and free and cued selective reminding test-delayed recall/trial 6.times. 100. In general, individuals with memory loss or MCI can have psychometric scores that are one or more standard deviation from the normal controls on one or more of the above measures. The controls can be age-and education-matched.

During the clinical interview, a battery of tests, including, but not limited to, the Mini-Mental State Exam, the Weschler Memory Scale, paired-associate learning, or any of the other tests mentioned above, are applied to diagnose memory loss. These tests provide general sensitivity to both general cognitive impairment and specific loss of learning/memory capacity. Apart from the specific diagnosis of dementia or amnesic disorders, these clinical instruments also identify age-related cognitive decline which reflects an objective diminution in mental function consequent to the aging process that is within normal limits given the person's age. The Weschler Memory Scale is a widely-used pencil-and-paper test of cognitive function and memory capacity. In the normal population with normal intellectual ability, the standardized test yields a mean of 100 and a standard deviation of 15, so that a mild amnesia can be detected with a 10-15 point reduction in the score, a more severe amnesia with a 20-30 point reduction, and so forth.

One illustrative declarative memory test for assessing the medial temporal lobe memory system is known as a Paragraph Recall Task. This task is a subtest of the Weschler Memory Scale—Revised. A subject is read a short story and then immediately asked to recall the story. After an approximate 30 minute retention interval, they are asked to recall the story again. The recall responses are scored for number of story items correctly recalled and there is a standard scoring system. There are two stories for each administration so the total score is the total of both stories combined; one for immediate memory; and one for delayed memory.

An illustrative working memory test is known as Subject Ordered Pointing (SOP). SOP is described, for example, in Janowsky et al., “Sex steroids modify working memory,” J Cogn Neuroscience 12(3):407-14, 2000 and Petrides et al., “Deficits on subject-ordered tasks after frontal- and temporal-lobe lesions in man,” Neuropsychologia 20:249-62, 1982. In general, subjects are presented with stacks of cards (6, 8, 10 or 12 cards/sets). Each card shows a regular array of abstract drawings, but the drawings are in a different spatial arrangement on each card. The subject is to touch one drawing on each card in any order, but not touch the same drawing on subsequent cards in the set. Subjects err when they touch a drawing that had been touched on a previous card in the set. Therefore, the subject has to remember previous drawings touched while planning a future response. Subjects repeat each 6, 8, 10, and 12 card set three times. The total number of errors across all card sets is the measure of interest. The examiner explains the task, then shows the subjects on two consecutive practice cards what would be the correct responses. Subjects are instructed to do the task at their own pace, and to try to not go too quickly or too slowly.

A further illustrative working memory test is known as the Trails test. This test is a dot-to-dot paper pencil test where the subject connects dots (1-2-3-4 etc.). On a second measure they connect dots but the dots are labeled alternately in letters and numbers so they have to shift their cognitive set and keep in mind where they are in each set: 1 to A to 2 to B to 3 etc. Their score is the number of seconds to complete the dot to dot.

As mentioned above, the methods described herein can also be used to treat amnesic disorder. Individuals with an amnesic disorder are impaired in their ability to learn new information or are unable to recall previously learned information or past events. The memory deficit is most apparent on tasks to require spontaneous recall and may also be evident when the examiner provides stimuli for the person to recall at a later time. The memory disturbance must be sufficiently severe to cause marked impairment in occupational functioning and must represent a significant decline from a previous level of functioning.

Dementia is a further condition that can be treated according to the methods disclosed herein. Dementia is characterized by multiple clinically significant deficits in cognition that represent a significant change from a previous level of functioning. Memory impairment involving inability to learn new material or forgetting of previously learned material is required to make the diagnosis of a dementia. Memory can be formally tested by asking the person to register, retain, recall and recognize information. The diagnosis of dementia also requires at least one of the following cognitive disturbances: aphasia, apraxia, agnosia or a disturbance in executive functioning. These deficits in language, motor performance, object recognition and abstract thinking, respectively, must be sufficiently severe in conjunction with the memory deficit to cause impairment in occupational or social functioning and must represent a decline from a previously higher level of functioning.

Improvement of a cognitive function, or amelioration of a cognitive dysfunction, can be discerned, for example, by determining if there is a statistically significant difference in the direction of normality in at least one cognitive function test. For instance, test results of the performance of treated patients may be compared to members of a placebo group or between subsequent tests given to the same patient.

The effectiveness of the treatment methods disclosed herein for a particular disorder can also be determined employing known animal model tests for cognitive function. More specifically, there are a variety of tests for cognitive function, especially learning and memory testing, which can be carried out using lesioned and normal animals. Learning and/or memory tests include, for example, inhibitory avoidance, contextual fear conditioning, visual delay non-match to sample, spatial delay non-match to sample, visual discrimination, Bames circular maze, Morris water maze and Radial arm maze tests.

An exemplary inhibitory avoidance test utilizes an apparatus that consists of a lit chamber that can be separated from a dark chamber by a sliding door. During training, the animal is placed in the lit chamber for some period of time, and then the door is opened. The animal moves to the dark chamber after a short delay, which is recorded (known as the latency). Upon entry into the dark chamber, the door is closed and a foot shock is delivered. Retention of the experience is determined after various time intervals, e.g., 24 or 48 hours, by repeating the test and recording the latency. The protocol is one of many variants of the inhibitory avoidance procedures (for review, see Rush, Behav Neural Biol 50:255, 1988).

An exemplary maze testing embodiment is the water maze working memory test. In general, the method utilizes an apparatus which consists of a circular water tank. The water in the tank is made cloudy by the addition of milk powder. A clear plexiglass platform, supported by a movable stand rest on the bottom of the tank, is submerged just below the water surface. Normally a swimming rat cannot perceive the location of the platform but it may recall it from a previous experience and training, unless it suffers from some memory impairment. The time taken to locate the platform is measured and referred to as the latency. During the experiment, all orientational cues such as ceiling lights etc. remain unchanged. Longer latencies are generally observed with rats with some impairment to their memory.

Another memory test includes the eyeblink conditioning test, which involves the administration of white noise or steady tone that precedes a mild air puff which stimulates the subject's eyeblink.

Still another memory test which can be used is fear conditioning, e.g., either “cued” and “contextual” fear conditioning. In one embodiment, a freeze monitor administers a sequence of stimuli (sounds, shock) and then records a series of latencies measuring the recovery from shock induced freezing of the animal.

Another memory test for the lesioned animals is a holeboard test, which utilizes a rotating holeboard apparatus containing (four) open holes arranged in a 4-corner configuration in the floor of the test enclosure. A mouse is trained to poke its head into a hole and retrieve a food reward from a “baited” hole which contains a reward on every trial. There is a food reward (e.g. Fruit Loop) in every exposed hole which is made inaccessible by being placed under a screen. The screen allows the odor of the reward to emanate from the hole, but does not allow access to the reinforcer. When an individual hole is baited, a small piece of Fruit Loop is placed on top of the screen, where it is accessible. The entire apparatus rests on a turntable so that it may be rotated easily to eliminate reliance on proximal (e.g. olfactory) cues. A start tube is placed in the center of the apparatus. The subject is released from the tube and allowed to explore for the baited (“correct”) hole.

Administration of Estrogenic Agents

In treating or preventing the above diseases, the estrogenic agents are administered in a therapeutically effective amount. The therapeutically effective amount will vary depending on the particular agent used and the route of administration, as is well known. The concentration of the estrogen agent is effective for delivery of an amount upon administration that lessens or ameliorates at least one symptom of the disorder for which the compound is administered. The concentration of active compound in the drug composition will depend on absorption, inactivation, and excretion rates of the active compound, the dosage schedule, and amount administered as well as other factors known to those of skill in the art.

For example, it has been found that cognitive impairment may be treated by providing in a subject an estrogenic serum concentration ranging from about 50 pg/ml to about 1500 pg/ml, more particularly about 100 pg/ml to about 1000 pg/ml for at least about four weeks.

In further examples, it has been found that cognitive impairment may be treated by administering an estrogenic agent in a dose of at least about 0.025, particularly 0.05, more particularly about 0. 1, most particularly about 0.2, and even more particularly about 0.5 mg/day. The maximum dosage, for example, may be about 1.5, particularly about 1.0, more particularly about 0.9, and most particularly about 0.7 mg/day.

The dose may be a single dose per day, it may be divided into at least two unit dosages for administration over a 24-hour period, or it may be a single continuous dose for a longer period of time, such as 1-10 weeks. In the example of transdermal administration via a patch(es) that is described below in more detail, the patch(es) may be applied daily, every three days, every seven days, or at other suitable intervals. Treatment may be continued as long as necessary to achieve the desired results. For instance, treatment may continue for about 3 or 4 weeks up to about 12-24 months, and possibly indefinitely.

The treatment disclosed herein involves administering to a patient in need of such treatment a pharmaceutical composition that includes a pharmaceutically acceptable carrier and a therapeutically-effective amount of the presently described compound. The compounds or pharmaceutical compositions typically are administered parenterally since oral administration of an estrogen agent to a male subject may be associated with increased risk of thromboembolic complications as well as with breast enlargement and tenderness. However, oral administration is not excluded from the methods described herein since certain strategies may be employed to ameliorate the side effects of oral administration. For instance, anticoagulants or anti-platelet agents can be co-administered to reduce thromboembolic conditions. Breast enlargement and tenderness in men can be partially prevented by a 3-day or 1-day course of radiation to the breasts prior to the start of estrogen-based therapy. Parenteral administration routes include, but are not limited to, subcutaneous injections (SQ and depo SQ), intravenous (IV), intramuscular (IM and depo-IM), intrasternal injection or infusion techniques, intranasal (inhalation), intrathecal, transdermal, topical, and ophthalmic. The drug may also be administered from implanted reservoirs or pumps, or it may be co-administered with other agents that are designed to treat prostate cancer or cognitive impairment. An example of another agent to treat cognitive impairment is a serotonin specific re-uptake inhibitor (SSRI) such as fluoxetine in a depressed subject with pseudodementia. Dosage forms known to those of skill in the art are suitable for delivery of the estrogen agent.

One or more estrogenic agents may be mixed or combined with a suitable pharmaceutically acceptable carrier to prepare pharmaceutical compositions. Upon mixing or addition of the agent(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the agent in the selected carrier or vehicle. The effective concentration is sufficient for lessening or ameliorating at least one symptom of the disease, disorder, or condition treated and may be empirically determined.

Pharmaceutical carriers or vehicles suitable for administration of the estrogenic agents include any such carriers known to be suitable for the particular mode of administration. In addition, the active materials can also be mixed with other active materials that do not impair the desired action, or with materials that supplement the desired action, or have another action. The agents may be formulated as the sole pharmaceutically active ingredient in the composition or may be combined with other active ingredients.

Where the agents exhibit insufficient solubility, methods for solubilizing may be used. Such methods are known and include, but are not limited to, using cosolvents such as dimethylsulfoxide (DMSO), using surfactants such as Tween® and dissolution in aqueous sodium bicarbonate. Derivatives of the estrogen agents, such as salts or prodrugs may also be used in formulating effective pharmaceutical compositions.

The estrogenic agents may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

The compounds and compositions can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for transdermal or topical administration.

Transdermal administration of the estrogenic agent is a particularly effective methodology, especially with respect to administering estradiol. Estradiol has limited oral effectiveness because it is rapidly metabolized by the liver to estrone and its conjugates, giving rise to higher circulating levels of estrone compared to estradiol. In contrast, the skin metabolizes estradiol only to a small extent. Therefore, transdermal administration produces therapeutic serum levels of estradiol with lower circulating levels of estrone and estrone conjugates.

In general, estrogen transdermal delivery patch systems include a skin-adhering substrate and an estrogen compound-containing formulation coated or incorporated into the substrate that can be released to the skin over a prolonged period (e.g., from 1 to 30 days). The patches typically are applied to a predetermined area of the skin. “Predetermined area” of skin or mucosal tissue refers to the area of skin or mucosal tissue through which a drug-enhancer formulation is delivered, and includes a defined area of intact unbroken living skin or mucosal tissue. That area will usually be in the range of about 5 cm² to about 200 cm², more usually in the range of about 5 cm² to about 100 cm², preferably in the range of about 20 cm² to about 60 cm². However, it will be appreciated by those skilled in the art of drug delivery that the area of skin or mucosal tissue through which the drug is administered may vary significantly, depending on patch configuration, dose, and the like.

Estrogen transdermal delivery systems are commercially available such as Climara® estradiol transdermal patch available from Berlex Laboratories, Estraderm® estradiol transdermal patch available from Novartis, Aerodiol estradiol hemihydrate nasal spray from Servier Laboratories, Alora from Watson Pharmaceutical, Esclim from Women First Healthcare, generic estradiol transdermal from Mylan, Vivelle from Novartis, and Vivelle-dot from Novartis.

The Climara® system includes (proceeding from the visible surface to the surface attached to the skin) a translucent polyethylene film and an acrylate adhesive matrix containing estradiol (specifically 17β-estradiol). A protective liner is attached to the adhesive surface and must be removed before the system can be used. Four different patch sizes of the Climara® system are available −6.5, 12.5, 18.75 and 25.0 cm² contact surface area—to provide nominal in vivo delivery of 0.025, 0.05, 0.075 or 0.1 mg respectively of estradiol per day. The 6.5, 12.5, 18.75 and 25.0 cm² patches contain 2.0, 3.8, 5.7 and 7.6 mg of estradiol, respectively. The period of continuous use for a single patch prior to removal typically is seven days.

U.S. Pat. No. 5,223,261 (Nelson et al.) describes the Climara® system and similar systems. According to Nelson et al., the system can also include (in addition to estradiol and an acrylic adhesive) a skin penetration enhancer combination that includes fatty acid esters such as isopropyl myristate, glyceryl monolaurate, and ethyl oleate. The acrylic adhesive, according to Nelson et al., is an acrylic polymer comprising at least about 91 to 98 percent by weight of a hydrophobic monomeric acrylic or methacrylic acid ester of an alkyl alcohol based on the weight of all monomers in the polymer or an acrylic copolymer comprising (i) about 60 to 80 percent by weight of a hydrophobic monomeric acrylic or methacrylic acid ester of an alkyl alcohol based on the weight of all of the monomers in the copolymer; (ii) about 4 to 9 percent by weight based on the weight of all of the monomers in the copolymer of a reinforcing monomer selected from acrylic acid, methacrylic acid, an alkyl acrylate or methacrylate containing 1 to 3 carbon atoms in the alkyl group, acrylamide, methacrylamide, a lower diacetone acrylamide, and a N-vinyl-2-pyrrolidone; and (iii) about 15 to 35 percent by weight of vinyl acetate based on the weight of all of the monomers in the copolymer. The estradiol is said to be present in an amount of about 0.2 to 12 weight percent of the total weight of the adhesive coating.

The Estraderm® patch system includes five layers. Proceeding from the visible surface toward the surface attached to the skin, these layers are (1) a transparent polyester backing film, (2) a drug reservoir of 17β-estradiol, (3) an ethylene vinyl acetate copolymer release-controlling membrane, (4) an adhesive formulation of light mineral oil and polyisobutylene, and (5) a protective liner of siliconized polyethylene terephthalate film attached to the adhesive surface for removal prior to use. Three different patch sizes of the Estraderm®) system are available—5, 10 and 20 cm² contact surface area—to provide nominal in vivo delivery of 25, 50 or 100 μg respectively of estradiol per day. The 5, 10 and 20 cm patches contain 2, 4, and 8 mg of estradiol, respectively. The period of continuous use for a single patch prior to removal typically is three or four days.

Injectable solutions or suspensions may be formulated, using suitable non-toxic, parenterally-acceptable diluents or solvents, such as mannitol; 1,3-butanediol; water; saline solution; Ringer's solution or isotonic sodium chloride solution; or suitable dispersing or wetting and suspending agents, such as sterile, bland, fixed oils, including synthetic mono- or diglycerides, and fatty acids, including oleic acid; a naturally occurring vegetable oil such as sesame oil, coconut oil, peanut oil, cottonseed oil, and the like; polyethylene glycol; glycerine; propylene glycol; or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required. Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. An intramuscular depot formulation of polyestradiolphosphate is commercially available as Estradurin from Pharmacia & Upjohn.

If the estrogenic agents are administered orally as a suspension, the pharmaceutical compositions may be prepared according to techniques well known in the art of pharmaceutical formulation and may contain microcrystalline cellulose for imparting bulk, alginic acid or sodium alginate as a suspending agent, methylcellulose as a viscosity enhancer, and sweeteners/flavoring agents. As immediate release tablets, these compositions may contain microcrystalline cellulose, dicalcium phosphate, starch, magnesium stearate and lactose and/or other excipients, binders, extenders, disintegrants, diluents and lubricants.

If oral administration is desired, the estrogenic agent should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

When administered orally, the compounds can be administered in usual dosage forms for oral administration. These dosage forms include the usual solid unit dosage forms of tablets and capsules as well as liquid dosage forms such as solutions, suspensions, and elixirs. When the solid dosage forms are used, it is preferred that they be of the sustained release type so that the compounds need to be administered only once or twice daily.

Suitable oral estrogen formulations are commercially available, for example, such as Estrofem® from Novo Nordisk (1 mg or 2 mg estradiol tablets), Premarin® from Wyeth (0.625 or 1.25 mg conjugated equine estrogen tablets), and Progynova® from Schering (1 mg or 2 mg estradiol valerate tablets).

In certain examples of the presently described therapies, the estrogenic agent is the only active agent that is administered for treating cognitive dysfunction.

In such combinations the compounds and other active agents may be administered separately or in conjunction. In addition, the administration of one agent may be prior to, concurrent to, or subsequent to the administration of other agent(s).

A useful method of monitoring the effect of a treatment for cognitive impairment is magnetic resonance imaging (“MRI”). Illustrative MRI methods include cell-specific imaging, magnetization transfer imaging (“MTI”), gadolinium-enhanced MRI, proton magnetic resonance spectroscopy (MRS), and diffusion-weighted imaging (DWI), functional MR imaging (fMRI).

It should be apparent to one skilled in the art that the exact dosage and frequency of administration will depend on the particular compounds administered, the particular condition being treated, the severity of the condition being treated, the age, weight, general physical condition of the particular patient, other medication the individual may be taking, and shin type or other characteristics known and unknown that might affect the transfer rate of estradiol from a patch.

### EXAMPLE 1

### Improvement in Cognitive Performance by Administering Estradiol to Patients with AIPC

Estradiol was transdermally administered to nineteen men with androgen-independent prostate cancer following a protocol outlined below.

Patients

Eligibility criteria included histologically or cytologically confirmed prostate cancer that is progressing despite standard hormonal therapy (i.e., androgen suppression therapy). Patients, whose primary hormonal therapy included an antiandrogen, must have discontinued the primary hormonal therapy (6 weeks for bicalutamide or nilutamide, 4 weeks for flutamide). Exclusion criteria included prior treatment for prostate cancer with chemotherapy, diethylstilbestrol or PC-SPES herbal preparation; major surgery within 4 weeks; significant cardiovascular illness; and active second malignancy other than non-melanoma skin cancer.

Treatment

Standard prophylactic breast irradiation was offered to all patients prior to therapy to reduce the risk of gynecomastia. Six Climara® estradiol patches (25.0 cm², 7.6 mg estradiol per patch) were adhered to the patients once every 7 days for a period of at least 4 weeks for a dosage of 0.6 mg estradiol/day (in vivo estradiol delivery per patch is 0.1 mg/day).

Monitoring

Certain cognitive functions of the patients were evaluated prior to starting estradiol treatment and approximately 4 weeks after starting estradiol treatment. Specifically, memory was assessed using immediate and delayed Paragraph Recall test and working memory was assessed using the SOP and Trails tests. Serum estradiol concentration was also measured after approximately 4 weeks of treatment, as well as other standard monitoring measurements. The Paragraph Recall test was performed as described above. The SOP test was performed as described above and was computer-administered with 8 and 10 card sets. Each card of the set was depicted on a computer screen and the subject selected the drawing for each card by pressing a corresponding number on the keyboard. The Trails test was performed as described above.

Results

The test results are shown below in Table 1.

| | Test | | |
|---|---|---|---|
| | Pre-treatment | On estrogen | P value |
| Paragraph Recall (immediate; | 19.2 | 22.9 | .007 |
| # correct) | | | |
| Paragraph Recall (delay; | 14.8 | 18.8 | .019 |
| # correct) | | | |
| Subject Ordered (# of errors) | | | |
| (data from 18 subjects) | | | |
| 8 item | 6.4 | 6.3 | NS |
| 10 item | 7.2 | 7.0 | NS |
| Trails (seconds B-A) | 66.0 | 69.9 | NS |
| (data from 15 subjects) | | | |

The evaluated men treated with estradiol surprisingly had a significant improvement in their long-term memory, but no change in working memory. In particular, the men remembered more elements of stories they had been told both immediately after hearing the story and after a half hour retention interval (the Paragraph Recall test). With respect to the immediate Paragraph Recall test, the men exhibited a 19% score improvement. With respect to the delayed Paragraph Recall test, the men exhibited a 27% score improvement. They showed no change in performance on the measure that requires the subject to hold information in mind and update, or on the measure of cognitive flexibility (the Subject-Ordered and Trails test). With respect to the Trails test, men with prostate cancer are slower than healthy men, but when corrected for speed they are not more impaired on working memory (the measure where they have to shift between numbers and letters) and the estrogen did not affect working memory.

There were no thromboembolic complications or other significant toxicity issues at the time of the most recent analysis. Two patients did not achieve elevated estradiol concentrations. In the remaining patients, the estradiol serum concentration ranged from 70 to 1045 pg/ml. Steroid hormone binding globulin (SHBG) was significantly increased with treatment. Hormone results are summarized in Table 2 below.

TABLE 2

| | Mean ± 95% CI | | | |
|---|---|---|---|---|
| | Baseline | 1 month | 2 months | P value |
| Estradiol (pg/ml) | 13.2 ± 3.4 | 460 ± 126 | 371 ± 115 | <0.0001 |
| SHBG (ug/dl) | 1.0 ± 0.2 | 2.7 ± 0.6 | 2.2 ± 0.4 | <0.0001 |

### EXAMPLE 2

### Further Testing of Estradiol Administration

Further testing was performed along the lines described above in Example 1 in order to (a) compare men that received estradiol with two control groups (men undergoing androgen deprivation therapy (ADT), and age-matched, healthy men), and (b) evaluate the effect of estradiol upon confusion and mood.

Patients and Treatment

Research subjects consisted of healthy men, and men who have been diagnosed with prostate cancer that were between the ages of 45-100. The subjects were classified into three groups: (1) Prostate cancer patients progressing on ADT and before and after starting second line therapy with transdermal estradiol as described above; (2) Prostate cancer patients continuing ADT; and (3) Age-matched healthy controls. The treatment regimen of these men was not modified as part of their participation in this study. Exclusion criteria included currently taking hormone replacement, active malignancy other than prostate cancer or non-melanoma skin cancer, major surgery within last 4 weeks, and score less than 25 on the Mini-Mental State Exam (a screening measure for dementia). The participant characteristics are shown below in Table 3.

TABLE 3

| | E | AD | HC* |
|---|---|---|---|
| | (n = 19) | (n = 17) | (n = 17) |
| Age | 70.7 (11.5) | 69.2 (11.0) | 63.2 (8.0) (ns) |
| Education | 14.8 (5.2) | 15.4 (2.2) | 15.8 (2.7) (ns) |
| MMSE | 26.9ₐ (2.5) | 27.9_{a, b} (1.2) | 28.5_{b} (1.0) p < .05 |
| WAIS-R^{†} | 45.5ₐ (5.8) | 50.1_{a, b} (7.3) | 53.4_{b} (6.6) p < .01 |

Note.
E = Estradiol;
AD = Androgen deprivation;
HC = Healthy Controls.
MMSE = Mini-Mental State Exam.
WAIS-R = Wechsler Adult Intelligence Scale - Revised.
^{†}Means having the same subscript are not significantly different at p < .05 in a Tukey least-significant difference comparison.

The estradiol administration was the same as in example 1.

Monitoring

The monitoring was similar as described above in connection with example 1. Certain cognitive functions of the patients were evaluated at the beginning of the study and then approximately 4 weeks later. In the case of patients receiving estradiol treatments, the patients were evaluated prior to the initial estradiol and then approximately 4 weeks after starting estradiol treatment. Verbal memory was assessed using the immediate Paragraph Recall test and working memory was assessed using the Trails tests. Serum estradiol concentration was also measured after approximately 4 weeks of treatment, as well as other standard monitoring measurements.

Mood was assessed using a Profile of Mood States (POMS) test as described in McNair et al, Profile of mood states manual (Education and Industrial Testing Service, San Diego (1971)). Participants completed the POMS test at both testing sessions (i.e., beginning of study and 4 weeks later). The scale asks participants to rate how they felt in the last week. Responses are categorized into six subscales (tension-anxiety, depression, anger, fatigue, confusion and vigor). Higher scores on the total composite score and the five negative mood sub-scales indicate more problems or greater negative effect. For the vigor subscale only, higher scores indicate greater well-being. Inclusion of this measure allowed for the examination of whether nonspecific effects of hormone administration on mood accounted for observed cognitive changes. With respect to confusion, participants indicate how much (not at all to extremely) they felt confused or bewildered. Sample items included confused, unable to concentrate, forgetful, and bewildered. With respect to depression, participants indicate how much they felt depressed/dejected. Sample items included unhappy, sad, and blue.

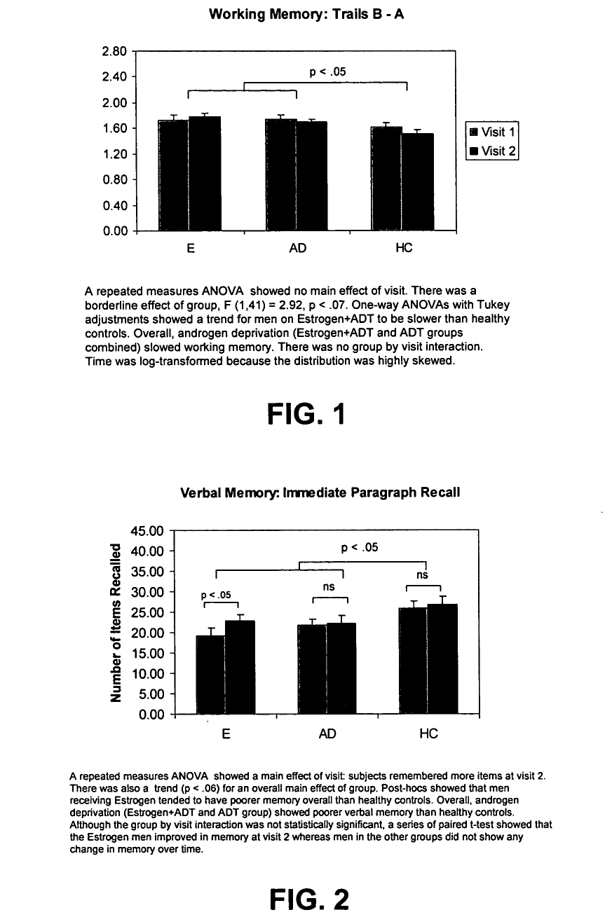

The serum concentration results are set forth in Table 4 and the cognitive test results are set forth in FIGS. 1-4.

TABLE 4
Sex steroids in men before and after
treatment with transdermal estradiol

| | Visit 1 | Visit 2 | p value |
|---|---|---|---|
| Estradiol | 18.3 (6.1) | 428.8 (280.7) | p < .001 |
| (pg/mL) | | | |
| SHGB | 34.4 (21.9) | 89.3 (39.8) | p < .001 |
| (nmol/dL) | | | |
| Total T (ng/dL) | 10.8 (6.9) | 9.7 (3.9) | ns |
| Free T (pmol/L) | 6.8 (4.9) | 3.5 (1.9) | p < .01 |

The working memory results (FIG. 1) indicate that ADT slowed working memory performance as assessed on the Trails test, but does not affect working memory accuracy (data not shown—from Subject Ordered pointing task). Estradiol therapy did not affect working memory. Androgen deprivation does not affect absolute speed of response (Trails A).

The verbal memory results (FIG. 2) indicate that ADT and/or prostate cancer significantly impaired verbal memory. Estradiol therapy, however, improved verbal memory performance.

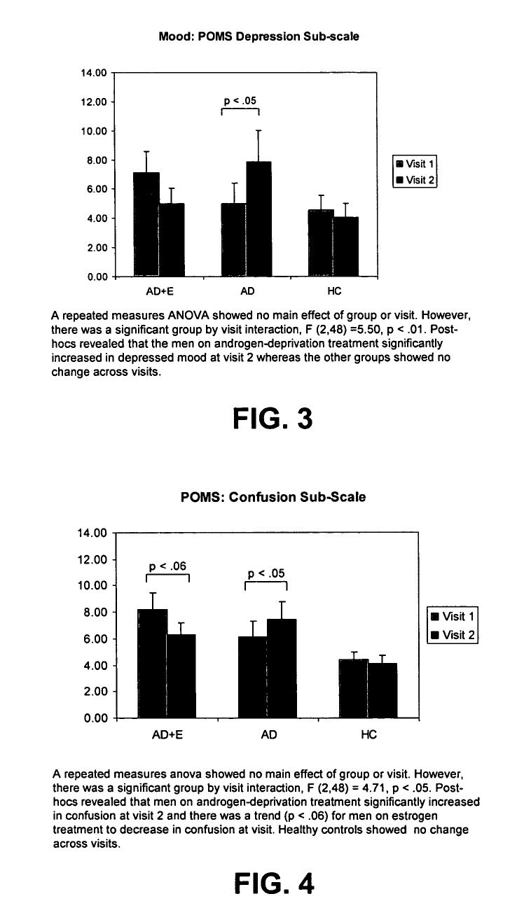

The mood and confusion results (FIGS. 3 and 4) indicate that men with prostate cancer on ADT exhibited increased depression and increased confusion when compared to healthy controls. However, there was a trend for men receiving estradiol to report a decrease in confusion.

Having illustrated and described the principles of the disclosed compounds, compositions and methods, it will be apparent that these compounds, compositions and methods may be modified in arrangement and detail without departing from such principles.

What is claimed is:

1. A method of treating a male subject diagnosed with cognitive dysfunction, comprising administering a therapeutically effective amount of an estrogenic agent, or a pharmaceutically acceptable salt or complex thereof, sufficient to achieve a supraphysiologic level of estrogenic agent in the serum of the male subject.

2. The method of claim 1, wherein the cognitive dysfunction is mild cognitive impairment or memory loss.

3. The method of claim 1, wherein the estrogenic agent comprises estradiol.

4. The method of claim 1, wherein the estrogenic serum concentration in the male subject is about 100 pg/ml to about 1000 pg/ml for a period of at least 4 weeks.

5. The method of claim 1, wherein the estrogenic agent, or the pharmaceutically acceptable salt or complex thereof, is administered at a dosage of about 0.025 to about 1.5 mg/day.

6. The method of claim 1, wherein the estrogenic agent is parenterally administered.

7. The method of claim 6, wherein the parenteral administration is via transdermal administration.

8. A method of improving cognitive or emotional function in a hypogonadal male subject, comprising transdermally administering to the male subject a therapeutically effective amount of an estrogenic agent, or a pharmaceutically acceptable salt or complex thereof.

9. The method of claim 8, comprising improving the memory of the male subject.

10. The method of claim 8, wherein the transdermal administration comprises applying at least one transdermal patch to a portion of the subject's body surface.

11. The method of claim 8, wherein the estrogenic agent is administered at a dosage of about 0.025 to about 1.5 mg/day.

12. The method of claim 8, wherein the hypogonadal male subject has not been subjected to androgen suppression therapy.

13. The method of claim 8, wherein the hypogonadal male subject has been subjected to androgen suppression therapy.

14. The method of claim 13, wherein the male subject has prostate cancer.

15. A method of improving cognitive or emotional function in a male subject, comprising administering to the male subject an estrogenic agent, or a pharmaceutically acceptable salt or complex thereof, at a dosage of about 0.025 to about 1.5 mg/day.

16. The method of claim 15, wherein the dosage is about 0.2 to about 1.0 mg/day.

17. The method of claim 15, wherein the male subject has age-related cognitive decline.
